(19) United States
(12) Patent Application Publication
(10) Pub. No.: US 2012/0157472 A1
(43) Pub. Date: Jun. 21, 2012

(54) METHOD FOR TREATING COLORECTAL CANCER

(75) Inventors: Annette Larsen, Paris (FR); Anke Baum, Vienna (AT); Frank Hilberg, Vienna (AT); Flavio Solca, Vienna (AT)

(73) Assignee: BOEHRINGER INGELHEIM INTERNATIONAL GMBH, Ingelheim am Rhein (DE)

(21) Appl. No.: 13/142,453

(22) PCT Filed: Jan. 13, 2010
(86) PCT No.: PCT/EP2010/050338

§ 371 Date: Dec. 19, 2011

(30) Foreign Application Priority Data
Jan. 14, 2009 (EP) 09150565.1

(51) Int. Cl. A61K 31/517; A61P 35/00

(52) U.S. Cl. 514/254.09

(57) ABSTRACT

The present invention relates to a method of treating patients suffering from colorectal cancer characterized by coadministration of BIBF 1120 and BIBW 2992, wherein in said method BIBF 1120 is administered according to a continuous daily regimen and BIBW 2992 is administered according to a weekly alternating on-off regimen, pharmaceutical compositions and kits suitable for this method as well as their preparation.

The present invention relates to a method of treating patients suffering from colorectal cancer comprising a flexible and active regimen for combining the triple angiogenesis inhibitor BIBF 1120 and the irreversible EGFR/HER2 inhibitor BIBW 2992, wherein this method BIBF 1120 is administered according to a continuous daily regimen and BIBW 2992 is co-administered according to a weekly on-off regimen to a patient in need of such treatment.

### BACKGROUND OF THE INVENTION

The efficacy of chemotherapeutic agents can be improved by using combination therapies with other chemotherapeutic, immunotherapeutic, immunomodulatory, antiangiogenic or hormonal compounds. Combination therapies constitute the gold standard in many settings of cancer therapy. Even if the concept of combining several therapeutic agents or therapies already has been suggested, and although various combination therapies are under investigation and in clinical trials, there is still a need for new and efficient therapeutic compositions for the treatment of cancer diseases, which show advantages over standard therapies.

Agents targeting EGFR or VEGF/VEGFR signaling are approved for treatment of metastatic colorectal cancer. Attempts to combine the two classes of agents have met limited success so far. BIBF 1120 (B1) is an orally active triple angiogenesis inhibitor targeting VEGFR, PDGFR and FGFR while BIBW 2992 (B2) is an orally active irreversible inhibitor of EGFR and HER2. The problem underlying this invention was to develop flexible regimens for combinations of B1 and B2 in colorectal cancer models with maximal activity and limited toxicity.

BIBF 1120 is known as the compound 3-Z-[1-(4-(N-((4-methyl-piperazin-1-yl)-methylcarbonyl)-N-methyl-amino)-anilino)-1-phenyl-methylene]-6-methoxycarbonyl-2-indolinone,

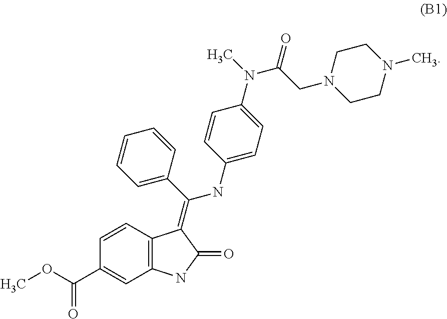

This compound is disclosed in WO 01/27081. Furthermore, the monoethanesulfonate salt of BIBF 1120 known from WO 04/13099 possesses properties which makes this salt form especially suitable for pharmaceutical use. The above mentioned patent applications further disclose the use of this compound or its monoethanesulfonate salt for the preparation of pharmaceutical compositions intended especially for the treatment of diseases characterized by excessive or abnormal cell proliferation.

BIBW2992 is known as the compound 4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline,

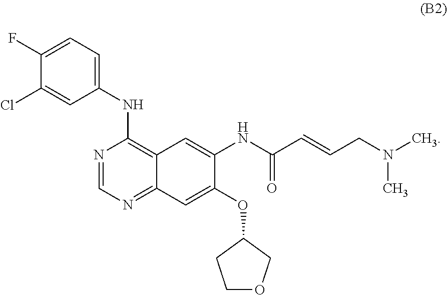

BIBW 2992 is a potent and selective dual inhibitor of erbb1 receptor (EGFR) and erbB2 (Her2/neu) receptor tyrosine kinases. Furthermore, BIBW 2992 was designed to covalently bind to EGFR and HER2 thereby irreversibly inactivating the receptor molecule it has bound to. This compound, salts thereof such as the dimaleate salt, their preparation as well as pharmaceutical formulations comprising BIBW 2992 or a salt thereof, indications to be treated with BIBW 2992 and combinations including BIBW 2992 are disclosed in WO 02/50043, WO 2005/037824, WO 2007/054550 and WO 2007/054551.

### SUMMARY OF THE INVENTION

A first object of the present invention is a method of treating patients suffering from colorectal cancer characterized by coadministration of effective amounts of BIBF 1120 and BIBW 2992 to a patient in need of such treatment, wherein said method BIBF 1120 is administered according to a continuous daily regimen and BIBW 2992 is administered according to a weekly alternating on-off regimen to a patient in need of such treatment.

A second object of the invention is a pharmaceutical composition comprising an effective amount of BIBF 1120 and BIBW 2992 together with an instruction for coadministration of both actives to a patient suffering from colorectal cancer, wherein according to said instruction BIBF 1120 is to be administered according to a continuous daily regimen and BIBW 2992 is to be administered according to a weekly alternating on-off regimen to the patient.

A third object of the invention is a pharmaceutical kit, comprising a first compartment which comprises an effective amount of BIBF 1120 and a second compartment which comprises BIBW 2992, together with an instruction for coadministration of both actives to a patient suffering from colorectal cancer, wherein according to said instruction BIBF 1120 is to be administered according to a continuous daily regimen and BIBW 2992 is to be administered according to a weekly alternating on-off regimen to the patient.

A fourth object of the present invention is the compound BIBF 1120 for its coadministration with BIBW 2992 to a patient suffering from colorectal cancer, characterized in that BIBF 1120 is administered according to a continuous daily regimen and BIBW 2992 is administered according to a weekly alternating on-off regimen to the patient.

A fifth object of the present invention is the use of BIBF 1120 for preparation of a pharmaceutical composition comprising an effective amount of BIBF 1120 and BIBW 2992 together with an instruction for coadministration of both actives to a patient suffering from colorectal cancer, wherein according to said instruction BIBF 1120 is to be administered according to a continuous daily regimen and BIBW 2992 is to be administered according to a weekly alternating on-off regimen to the patient.

A sixth object of the present invention is the use of BIBF 1120 for preparation of a pharmaceutical kit, comprising a first compartment which comprises an effective amount of BIBF 1120 and a second compartment which comprises BIBW 2992, together with an instruction for coadministration of both actives to a patient suffering from colorectal cancer, wherein according to said instruction BIBF 1120 is to be administered according to a continuous daily regimen and BIBW 2992 is to be administered according to a weekly alternating on-off regimen to the patient.

### DETAILED DESCRIPTION OF THE INVENTION

The continuous daily regimen to be used for the administration of BIBF 1120 means that a daily dosage of BIBF 1120 or a pharmaceutically acceptable salt thereof is administered to the patient in need thereof.

The weekly alternating on-off regimen to be used for the administration of BIBW 2992 means that a daily dosage of BIBW 2992 or a pharmaceutically acceptable salt thereof is administered to the patient in need thereof for a week, followed by a week of recovery without administering this active, on an alternating basis.

In a first sub-regimen the weekly alternating on-off regimen includes that a daily dosage of the active is administered to the patient in need thereof only on the days 1, 3, 5 and 7 for a week, followed by a week of recovery without administering this active, on an alternating basis.

In a second sub-regimen the weekly alternating on-off regimen includes that a daily dosage of the active is administered to the patient in need thereof only on the days 2, 4, and 6 for a week, followed by a week of recovery without administering this active, on an alternating basis.

In a third sub-regimen the weekly alternating on-off regimen includes that a daily dosage of the active is administered to the patient in need thereof only on the days 1 and 7 for a week, followed by a week of recovery without administering this active, on an alternating basis.

In a fourth sub-regimen the weekly alternating on-off regimen includes that a daily dosage of the active is administered to the patient in need thereof only on the days 2 and 6 for a week, followed by a week of recovery without administering this active, on an alternating basis.

In a fifth sub-regimen the weekly alternating on-off regimen includes that a daily dosage of the active is administered to the patient in need thereof only on the days 3 and 5 for a week, followed by a week of recovery without administering this active, on an alternating basis.

In a sixth sub-regimen the weekly alternating on-off regimen includes that a daily dosage of the active is administered to the patient in need thereof only on one of days 1, 2, 3, 4, 5, 6 or 7 for a week, followed by a week of recovery without administering this active, on an alternating basis.

The instruction for coadministration may be in any form suitable for pharmaceuticals, e.g. in form of a leaflet added to the dosage form within secondary packaging or an imprint on the primary or secondary packaging.

### Dosages/BIBW 2992:

For oral treatment BIBW 2992 may be administered to the human patient in a daily dose of (0.01-4 mg/kg of body weight (bw), preferably 0.1-2 mg/kg, particularly preferred in a dose of 0.2-1.3 mg/kg bw. For instance, BIBW 2992 may be administered orally in a total daily dose of 10, 20, 30, 40, 50, 60, 70, 100 or 150 mg, optionally divided into multiple doses, e.g. 1, 2 or 3 doses to be administered through the day. Preferably the oral daily dose is administered only once a time.

The dosage for intravenous use of BIBW 2992 may be 1-500 mg, preferably 5-300 mg, particularly preferred 10-100 mg, either given as a bolus or, especially if higher doses are applied, as a slow intravenous infusion over several hours, e.g. over about 1, 2, 4, 6, 10, 12 or 24 hours.

### Dosages/BIBF 1120:

In the context of the invention BIBF 1120 is administered in a daily dosage such that the maximum plasma concentration in the plasma of human subjects preferably is within a range of 4 ng/ml and 32 ng/ml, if a dosage form comprising 150 mg (3 times 50 mg) of BIBF 1120 monoethanesulphonate has been administered.

For oral treatment BIBF 1120 may be administered daily in a total dose of 10 to 300 mg, e.g 20, 30, 40, 50, 60, 70, 100, 150 mg one or two times daily or 200, 225, 250, 275 or 300 mg once a day. The total daily dose may also be divided into three subdoses to be taken within one day. Preferably, the oral daily dose is administered in two subdoses, e.g. each of 100 mg.

However, it may optionally be necessary to deviate from the dosage amounts specified for BIBW 2992 and BIBF 1120, depending on the body weight or method of administration, the individual response to the medication, the nature of the formulation used and the time or interval over which it is administered. Thus, in some cases, it may be sufficient to use less than the minimum quantity specified above, while in other cases the upper limit specified will have to be exceeded. When large amounts are administered it may be advisable to spread them over the day in a number of single doses.

### Dosage Forms and Formulation Aspects

Regarding any aspects of the invention pharmaceutically acceptable salts of the actives may be used, preferably BIBF 1120 monoethanesulphonate and BIBW2992 dimaleinate. Dosages or amounts of the actives provided in the context of this invention refer in any case to the free base equivalent, that is BIBF 1120 and BIBW 2992 in the free base form.

The term “therapeutically effective amount” shall mean that amount of a drug or pharmaceutical agent that will elicit the biological or medical response of a tissue system, animal or human that is being sought by a researcher or clinician, resulting in a beneficial effect for at least a statistically significant fraction of patients, such as a improvement of symptoms, a cure, a reduction in disease load, reduction in tumor mass or cell numbers, extension of life, or improvement in quality of life.

In accordance with the present invention BIBW 2992 and BIBF 1120 may be administered by oral (including buccal or sublingual), enterical, parenteral (e.g. intramuscular, intraperitoneal, intravenous, transdermal or subcutaneous injection, or implant), nasal, vaginal, rectal, or topical (e.g. inhalative) routes of administration and may be formulated, alone or together, in suitable dosage unit formulations containing conventional non-toxic pharmaceutically acceptable carriers, adjuvants and vehicles appropriate for each route of administration.

In a preferred embodiment BIBW 2992 and BIBF 1120 are administered orally, enterically, transdermally, intravenously, peritoneally or by injection, preferably orally. Dosage forms and formulations of both actives suitable within the present invention are known in the art. For instance, such dosage forms and formulations include those disclosed for BIBW 2992 in WO 02/50043, WO 2007/054550 and WO 2007/054551 and those disclosed for BIBF 1120 in WO 01/27081, WO 2004/013099 and WO2007/141283.

The following Examples serve to illustrate the invention without restricting it:

### EXAMPLE 1

### Biological Tests

Methods.

A panel of 14 well-characterized human colorectal cancer cell lines were used to characterize the in vitro effects of BIBF 1120 (B1), BIBW 2992 (B2) and their combinations. The antitumor effects of the 2 drugs were evaluated in mice with HT-29 colorectal xenografts.

Results.

Among the different combinations studied, continuous B1 with administration of B2 every second week proved almost as efficient in terms of tumor growth inhibition as continuous B1 and B2 together and was clearly superior to either agent alone. Both B1 and B2 inhibited angiogenesis, as demonstrated by CD31 staining, although by different mechanisms. Exposure of colorectal cancer cells to B1 or B2 was accompanied by growth inhibition linked to extensive G1 arrest, which in some cell lines was accompanied by delayed apoptosis. Strikingly, combinations of B1 and B2 were accompanied by increased cell death in all 6 cellular models studied.

Conclusions.

Both B1 and B2 have cytotoxic as well as antiangiogenic activity. B1-B2 combinations show more than additive antitumor effects. Continuous B1 with B2 every second week was identified as an active regimen with flexibility for addition of cytotoxic agents, such as disclosed in WO 2007/054551. Thus it can be expected that at least the same therapeutic effect will be obtained by the method of treatment according to the invention compared to continuous cotreatment with both actives, bearing the advantage of reduced total dosages to be administered to the patient.

### Legend to the Figures

FIG. 1: B1 and B2 show activity in the HT-29 CRC xenograft model.

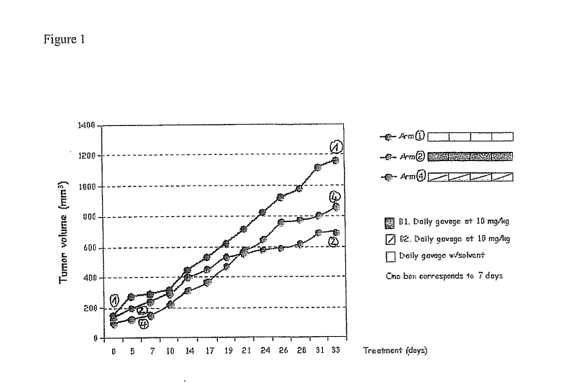

The tumor growth inhibitory activities of B1 and B2 were determined for HT-29 xenografts after daily drug administration (10 mg/kg p.o.) or, for control animals, by oral administration of the solvent. FIG. 1 shows development of the tumor volume [mm³] over treatment time [days]. The results show that both agents exhibit growth inhibitor activity toward HT-29 xenografts. Since the studies were designed to detect additive or synergistic interactions between the two drugs in subsequent experiments, only sub-optimal doses were used in these studies. Each group represents at least seven mice.

FIG. 2: Antitumor activity of B1-B2 combinations.

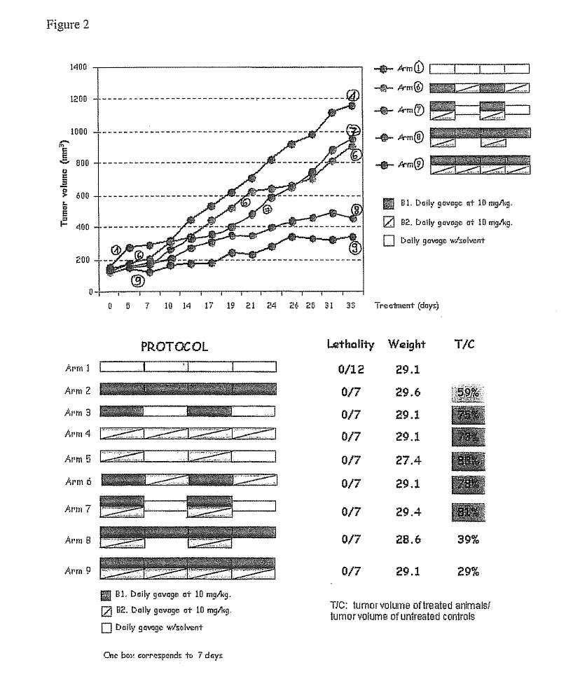

The tumor growth inhibitory activities of B1 and B2 were determined for HT-29 xenografts after daily drug administration (10 mg/kg p.o.) or, for control animals, by oral administration of the solvent.

Arm 1 shows the control,

arm 6 shows the results obtained with a weekly alternating treatment regime of one week B1 (10 mg/kg daily)-one week B2 (10 mg/kg daily),

arm 7 shows the results obtained with a weekly alternating treatment regime of one week B1-B2 combination (both 10 mg/kg daily)-one week off treatment,

arm 8 shows the results obtained with a treatment regime of continuous B1 (10 mg/kg daily) combined with B2 every second week (10 mg/kg daily), according to the invention,

arm 9 shows the results obtained with continuous B1-B2 co-treatment (both 10 mg/kg daily).

The results reveal modest activity of sequential B1-B2 combination, as well as of the “one week on-one week off” protocol where B1 and B2 were administered simultaneously every second week. In clear contrast, continuous B1 administration with B2 every second week was almost as active as continuous administration of the two drugs together. No lethality or weight loss was observed for any of the treatment groups. In conclusion, continuous B1 with B2 every second week was identified as an active regimen with flexibility for addition of cytotoxic agents.

### EXAMPLE 2

### Pharmaceutical Compositions of Solid BIBW 2992 MA₂ Tablets

### (MA2: Dimaleinate)

TABLE 1

| | | Formulation | | | | |
|---|---|---|---|---|---|---|
| | | A | B | C | D | E |
| | % per | mg per | mg per | mg per | mg per | mg per |
| Ingredient | tablet | tablet | tablet | tablet | tablet | tablet |
| BIBW 2992 MA₂, un-milled | 16.42 | 29.5600 | 44.3400 | 59.1200 | 73.9000 | 103.4600 |
| (corresponding to BIBW | | (20.0000) | (30.0000) | (40.0000) | (50.0000) | (70.0000) |
| 2992 base) | | | | | | |
| Lactose monohydrate | 68.81 | 123.8603 | 185.7904 | 247.7206 | 309.6508 | 433.5110 |
| Microcrystalline cellulose | 10.27 | 18.4797 | 27.7196 | 36.9594 | 46.1993 | 64.6790 |
| Crospovidone | 2.00 | 3.6000 | 5.4000 | 7.2000 | 9.0000 | 12.6000 |
| Colloidal anhydrous silica* | 0.50 | 0.9000 | 1.3500 | 1.8000 | 2.2500 | 3.1500 |
| Magnesium stearate | 2.00 | 3.6000 | 5.4000 | 7.2000 | 9.0000 | 12.6000 |
| Total | 100.00 | 180.0000 | 270.0000 | 360.0000 | 450.0000 | 630.0000 |

Formulations A, B and C, D and E are tablets which can be coated with a film-coat according to Table 2.

TABLE 2
Exemplary composition of filmcoatings for formulation A-E

| | Coating for Formulation | | | | |
|---|---|---|---|---|---|
| | A | B | C | D | E |
| Ingredient | mg per tablet | | | | |
| Hypromellose | 2.5000 | 3.5000 | 4.0000 | 5.0000 | 6.0000 |
| Polyethylene glycol | 0.5000 | 0.7000 | 0.8000 | 1.0000 | 1.2000 |
| 400 | | | | | |
| Titanium dioxid | 1.1300 | 0.6825 | 1.8080 | 0.9750 | 1.1700 |
| Indigo Carmine | 0.0700 | 0.2450 | 0.1120 | 0.3500 | 0.4200 |
| aluminum lacquer | | | | | |
| Talcum | 0.6500 | 1.6625 | 1.0400 | 2.3750 | 2.8500 |
| Polysorbate 80 | 0.1500 | 0.2100 | 0.2400 | 0.3000 | 0.3600 |
| Purified water | — | — | — | — | — |
| (volatile component) | | | | | |
| Total | 5.0000 | 7.0000 | 8.0000 | 10.0000 | 12.0000 |

### EXAMPLE 3

### Pharmaceutical Compositions of BIBF 1120 Monoethanesulfonate Capsules

TABLE 3
Soft gelatin capsule containing 50 mg of active substance

| | | Formulation A | Formulation B | Formulation C |
|---|---|---|---|---|
| Ingredients | Function | mg per capsule | mg per capsule | mg per capsule |
| Active Substance | Active | 60.20 | 60.20 | 60.20 |
| | Ingredient | | | |
| Triglycerides, | Carrier | 40.95 | 53.70 | 54.00 |
| Medium-chain | | | | |
| Hard fat | Thickener | 38.25 | 25.50 | 25.50 |
| Lecithin | Wetting | 0.60 | 0.60 | 0.30 |
| | agent/ | | | |
| | Glidant | | | |
| Gelatin | Film-former | 72.25 | 72.25 | 72.25 |
| Glycerol 85% | Plasticizer | 32.24 | 32.24 | 32.24 |
| Titanium dioxide | Colorant | 0.20 | 0.20 | 0.20 |
| Iron oxide A | Colorant | 0.32 | 0.32 | 0.32 |
| Iron oxide B | Colorant | 0.32 | 0.32 | 0.32 |
| Total Capsule | | 245.33 | 245.33 | 245.33 |
| Weight | | | | |

1. A method of treating patients suffering from colorectal cancer characterized by coadministration of effective amounts of BIBF 1120, or a pharmaceutically acceptable salt thereof, and BIBW 2992, or a pharmaceutically acceptable salt thereof, to a patient in need of such treatment, wherein said method BIBF 1120 is administered according to a continuous daily regimen and BIBW 2992 is administered according to a weekly alternating on-off regimen to a patient in need of such treatment.

2. (canceled)

3. A pharmaceutical kit, comprising a first compartment which comprises an effective amount of BIBF 1120, or a pharmaceutically acceptable salt thereof, and a second compartment which comprises BIBW 2992, or a pharmaceutically acceptable salt thereof, together with an instruction for coadministration of both actives to a patient suffering from colorectal cancer, wherein according to said instruction BIBF 1120 is to be administered according to a continuous daily regimen and BIBW 2992 is to be administered according to a weekly alternating on-off regimen to the patient.

4-6. (canceled)

7. A method of treating patients suffering from colorectal cancer comprising administering to a patient in need of such treatment, the pharmaceutical kit according to claim 3.

8. The method according to claim 1 where the weekly alternating on-off regimen to be used for the administration of BIBW 2992 or a pharmaceutically acceptable salt wherein a daily dosage of the active is administered to the patient in need thereof only on the days 1, 3, 5 and 7 for a week, followed by a week of recovery without administering this active, on an alternating basis.

9. The method according to claim 1 where the weekly alternating on-off regimen to be used for the administration of BIBW 2992 or a pharmaceutically acceptable salt wherein a daily dosage of the active is administered to the patient in need thereof only on the days 2, 4, and 6 for a week, followed by a week of recovery without administering this active, on an alternating basis.

10. The method according to claim 1 where the weekly alternating on-off regimen to be used for the administration of BIBW 2992 or a pharmaceutically acceptable salt wherein a daily dosage of the active is administered to the patient in need thereof only on the days 1 and 7 for a week, followed by a week of recovery without administering this active, on an alternating basis.

11. The method according to claim 1 where the weekly alternating on-off regimen to be used for the administration of BIBW 2992 or a pharmaceutically acceptable salt wherein a daily dosage of the active is administered to the patient in need thereof only on the days 2 and 6 for a week, followed by a week of recovery without administering this active, on an alternating basis.

12. The method according to claim 1 where the weekly alternating on-off regimen to be used for the administration of BIBW 2992 or a pharmaceutically acceptable salt wherein a daily dosage of the active is administered to the patient in need thereof only on the days 3 and 5 for a week, followed by a week of recovery without administering this active, on an alternating basis.

13. The method according to claim 1 where the weekly alternating on-off regimen to be used for the administration of BIBW 2992 or a pharmaceutically acceptable salt includes that a daily dosage of the active is administered to the patient in need thereof only on one of days 1, 2, 3, 4, 5, 6 or 7 for a week, followed by a week of recovery without administering this active, on an alternating basis.

14. The method according to any one of claims 8-13 wherein
for oral treatment BIBW 2992 is administered orally in a total daily dose of 10, 20, 30, 40, 50, 60, 70, 100 or 150 mg, optionally divided into multiple doses;
or for intravenous use of BIBW 2992 is administered 10-100 mg, as a intravenous infusion over about 1, 2, 4, 6, 10, 12 or 24 hours;
BIBF 1120 is orally administered 20, 30, 40, 50, 60, 70, 100, 150 mg one or two times daily or 200, 225, 250, 275 or 300 mg once a day.
